(19) United States
(12) Patent Application Publication
(10) Pub. No.: US 2014/0303258 A1
(43) Pub. Date: Oct. 9, 2014

(54) Method of solubilizing insoluble bioactive compounds using triterpene glycosides and compositions thereof

(71) Applicants: Muhammed Majeed, Edison, NJ (US); Reza Kamarei, East Windsor, NJ (US); Beena Bhat, Bangalore (IN); Natarajan Sankaran, Bangalore (IN)

(72) Inventors: Muhammed Majeed, Edison, NJ (US); Reza Kamarei, East Windsor, NJ (US); Beena Bhat, Bangalore (IN); Natarajan Sankaran, Bangalore (IN)

(21) Appl. No.: 14/244,902

(22) Filed: Apr. 4, 2014

Related U.S. Application Data
(60) Provisional application No. 61/808,744, filed on Apr. 5, 2013.

(51) Int. Cl. A61K 47/26; A61K 31/12

(52) U.S. Cl. CPC A61K 47/26; A61K 31/12
USPC 514/679

(57) ABSTRACT

Disclosed are the uses of triterpene glycosides as non-toxic natural solubilizing agents for bioactive compounds and/or metabolites thereof. Triterpene glycosides are also shown to solubilize insoluble natural extracts. Also disclosed are exemplary methods and compositions incorporating the uses of triterpene glycosides as non-toxic natural solubilizing agents.

### CROSS REFERENCE TO RELATED APPLICATIONS

This application is a non-provisional filing of U.S. provisional patent application No. 61/808,744 filed on 5 Apr. 2013.

### BACKGROUND OF THE INVENTION

1. Field of the Invention

The present invention pertains to the uses of triterpene glycosides as non-toxic natural solubilizing agents for bioactive compounds and metabolites thereof.

2. Description of Prior Art

Poor aqueous solubility is a common technical difficulty encountered in formulating many water insoluble bioactive compounds. Terpene glycosides as naturally occurring class of water solubility enhancers is known in prior art. Diterpene glycosides like rubusoside, rebaudioside, steviol monoside and stevioside and their application in solubilising curcuminoids has been documented in US20110033525 by Liu, Zhijun. However, there exists the technical problem in the art that diterpene glycosides do not bring the water insoluble bioactives into solution in any considerable quantities. The increases in solubility achieved are very minimal and probably without any practical value. The present invention presents the unexpected finding that triterpene glycosides behave quite differently and bring into aqueous solution, water insoluble bioactives by increasing the solubility, in some cases, several thousand times. The methods and compositions of the present invention thus solve the technical difficulties inherent in US20110033525. More specifically, the triterpene glycosides of the present invention brings water insoluble bioactive compounds into the aqueous phase via formation of either a clear solution (where molecules of bioactive compounds freely tumble in the solvent) or an opaque/turbid emulsion (where molecules of bioactive compounds are engulfed by triterpene glycosides). Further, the triterpene glycosides of the instant invention are selectively chosen in such a way that the terpenic segment will synergize with the bioactive's pharmacological activity and also enhance the stability of the bioactive compound in terms of its longer lasting biological activity.

Accordingly, it is the principle objective of the present invention to use triterpene glycosides as non-toxic natural solubilizing agents for bioactive compounds and metabolites thereof.

The present invention fulfills the aforesaid objective and provides further related advantages.

### SUMMARY OF THE INVENTION

Disclosed are the uses of triterpene glycosides as non-toxic natural solubilizing agents for bioactive compounds and metabolites thereof. Also disclosed are exemplary methods and compositions incorporating the uses of triterpene glycosides as non-toxic natural solubilizing agents for bioactive compounds and metabolites thereof.

### DESCRIPTION OF THE FIGURES

FIG. 1 shows the solubilisation of curcuminoids (Curcumin C3 Complex®) using Bacopa glycosides including 25% w/w to 75% w/w of total Bacosides (Bacopin®) and Centella glycosides including at least 8% w/w of total triterpenes (Centellin®).

FIGS. 2, 2A and 2B show the curcuminoids and Bacopa glycosides emulsion reconstituted by adding water to the previously dried curcuminoids-Bacopa glycosides emulsion.

FIGS. 3A, 3B, 3C, 3D and 3E show the solubilisation of bioactive compounds forskolin, boswellic acids and pterostilbene, saw palmetto extract and ginger soft extract using Bacopa glycosides including 25% w/w to 75% w/w of total Bacosides (Bacopin®).

FIG. 4 shows the solubilisation of tetrahydrocurcuminoids using Bacopa glycosides including 25% w/w to 75% w/w of total Bacosides (Bacopin®).

### DETAILED DESCRIPTION OF THE MOST PREFERRED EMBODIMENT

In the most preferred embodiment, the present invention relates to a method of solubilizing water insoluble bioactive compounds or metabolites thereof using triterpene glycosides and compositions obtained thereof. In specific embodiments, the triterpene glycosides include one selected from the group comprising Bacopa glycosides obtained from Bacopa monnieri, Centella glycosides from Centella Asiatica and Shatavari glycosides obtained from Asparagus racemosus. In further specific embodiments, the insoluble bioactive compounds include curcuminoids, boswellic acids, forskolin, Resveratrol and pterostilbene. In an alternate most preferred embodiment, the present invention also relates to a method of solubilizing water insoluble extracts from natural sources like Saw Palmetto extract and Ginger extract and compositions obtained thereof.

In a preferred embodiment, the present invention relates to method of making water soluble curcuminoids or metabolites thereof using natural triterpene glycosides as solubilizing agents and compositions obtained thereof. In further specific embodiments the metabolites of curcuminoids include Phase I & Phase II metabolites. In further specific embodiments the metabolites of curcuminoids include tetrahydrocurcumin, tetrahydrodemethoxycurcumin, tetrahydrobisdemethoxycurcumin, hexahydrocurcumin and octahydrocurcumin, their glucuronides and sulphates, and in addition to degraded metabolites such as ferulic acid, feruloyl methane and also oxidized metabolites such as Biclyclopentadione. In still further specific embodiments, the natural triterpene glycosides is one selected from the group comprising Bacopa glycosides obtained from Bacopa monnieri, Centella glycosides from Centella Asiatica and Shatavari glycosides obtained from Asparagus racemosus. Still further, the Shatavari glycosides are standardized for not less than 4% w/w (HPLC) Shatavarin IV, the Bacopa glycosides to include 25% w/w to 75% w/w of total Bacosides and the Centella glycosides include at least 8% w/w of total triterpenes.

In another preferred embodiment, the present invention relates to method of making water soluble forskolin using natural triterpene glycosides as solubilizing agents and compositions obtained thereof. In still further specific embodiments, the natural triterpene glycosides is include one selected from the group comprising Bacopa glycosides obtained from Bacopa monnieri, Centella glycosides from Centella Asiatica and Shatavari glycosides obtained from Asparagus racemosus. Still further, the Shatavari glycosides are standardized for not less than 4% w/w (HPLC) Shatavarin IV, the Bacopa glycosides to include 25% w/w to 75% w/w of total Bacosides and the Centella glycosides include at least 8% w/w of total triterpenes.

In yet another preferred embodiment, the present invention relates to method of making water soluble boswellic acids using natural triterpene glycosides as solubilizing agents and compositions obtained thereof. In still further specific embodiments, the natural triterpene glycosides is include one selected from the group comprising Bacopa glycosides obtained from Bacopa monnieri, Centella glycosides from Centella Asiatica and Shatavari glycosides obtained from Asparagus racemosus. Still further, the Shatavari glycosides are standardized for not less than 4% w/w (HPLC) Shatavarin IV, the Bacopa glycosides to include 25% w/w to 75% w/w of total Bacosides and the Centella glycosides include at least 8% w/w of total triterpenes.

In still further preferred embodiment, the present invention relates to method of making water soluble resveratrol and its dimethylated analog pterostilbene using natural triterpene glycosides as solubilizing agents and compositions obtained thereof. In still further specific embodiments, the natural triterpene glycosides is include one selected from the group comprising Bacopa glycosides obtained from Bacopa monnieri, Centella glycosides from Centella Asiatica and Shatavari glycosides obtained from Asparagus racemosus. Still further, the Shatavari glycosides are standardized for not less than 4% w/w (HPLC) Shatavarin IV, the Bacopa glycosides to include 25% w/w to 75% w/w of total Bacosides and the Centella glycosides include at least 8% w/w of total triterpenes.

In alternate preferred embodiments, the present invention also relates to a method of making water soluble Saw palmetto and ginger extracts using natural triterpene glycosides as solubilizing agents and compositions obtained thereof. In still further specific embodiments, the natural triterpene glycosides is include one selected from the group comprising Bacopa glycosides obtained from Bacopa monnieri, Centella glycosides from Centella Asiatica and Shatavari glycosides obtained from Asparagus racemosus. Still further, the Shatavari glycosides are standardized for not less than 4% w/w (HPLC) Shatavarin IV, the Bacopa glycosides to include 25% w/w to 75% w/w of total Bacosides and the Centella glycosides include at least 8% w/w of total triterpenes.

The following examples are included herein as exemplary embodiments of the present invention.

### EXAMPLE 1

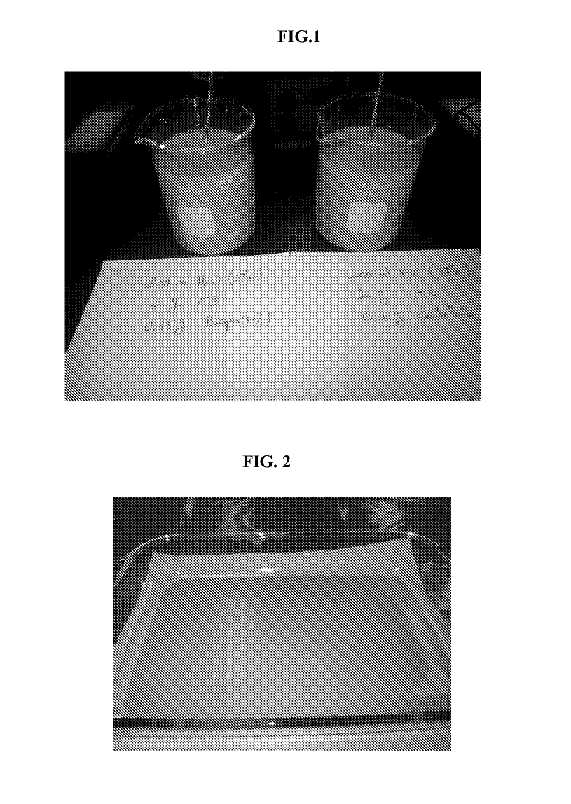

A. Two beakers each containing 200 ml distilled water (37° C.) were taken.
B. To each beaker, 2 grams commercially available curcuminoids (Curcumin C3 Complex®) was added and gently mixed. Curcuminoids essentially remained on the top and did not go into solution.
C. To the first beaker, 0.35 gram of Bacopa glycosides including 25% w/w to 75% w/w of total Bacosides (Bacopin®) was added and gently mixed for about 15 minutes. This resulted in a homogenous emulsion (See FIG. 1). This corresponded to a Critical Micelle Concentration (CMC) of 17.5%
D. To the second beaker, 0.40 gram of Centella glycosides including at least 8% w/w of total triterpenes (Centellin®) was added and gently mixed for about 15 minutes. This resulted in a homogenous emulsion (FIG. 1). This corresponded to Critical Micelle Concentration (CMC) of 20.0%.

### EXAMPLE 2

The compositions obtained in Example 1 namely curcuminoids-Bacopa glycosides emulsion and curcuminoids-Centella glycosides emulsion were stored in refrigerator (4° C.). for 10 days. The physical property of the solution was the same as the first day: some soft settlement (not precipitating out) of micelles which went easily back into an opaque/turbid solution upon mixing went was seen.

### EXAMPLE 3

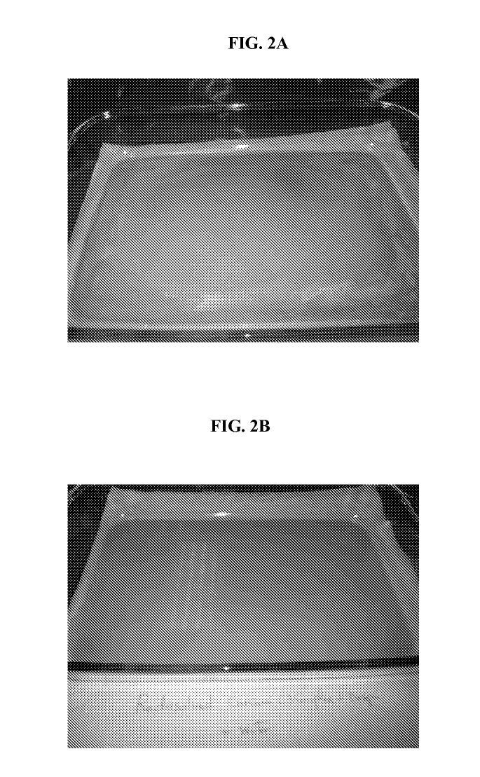

A. 100 ml of curcuminoids-Bacopa glycosides emulsion obtained in Example 1 was poured into a large glass container and allowed to dry at room temperature (FIG. 2).
B. After two days at room temperature, the curcuminoids-Bacopa glycosides emulsion was completely dry (FIG. 2A).
C. 100 ml of water at 37° C. water was added back to the dried curcuminoids-Bacopa glycosides emulsion and mixed it to uniformity. The new emulsion was identical to the original emulsion (FIG. 2B).

### EXAMPLE 4

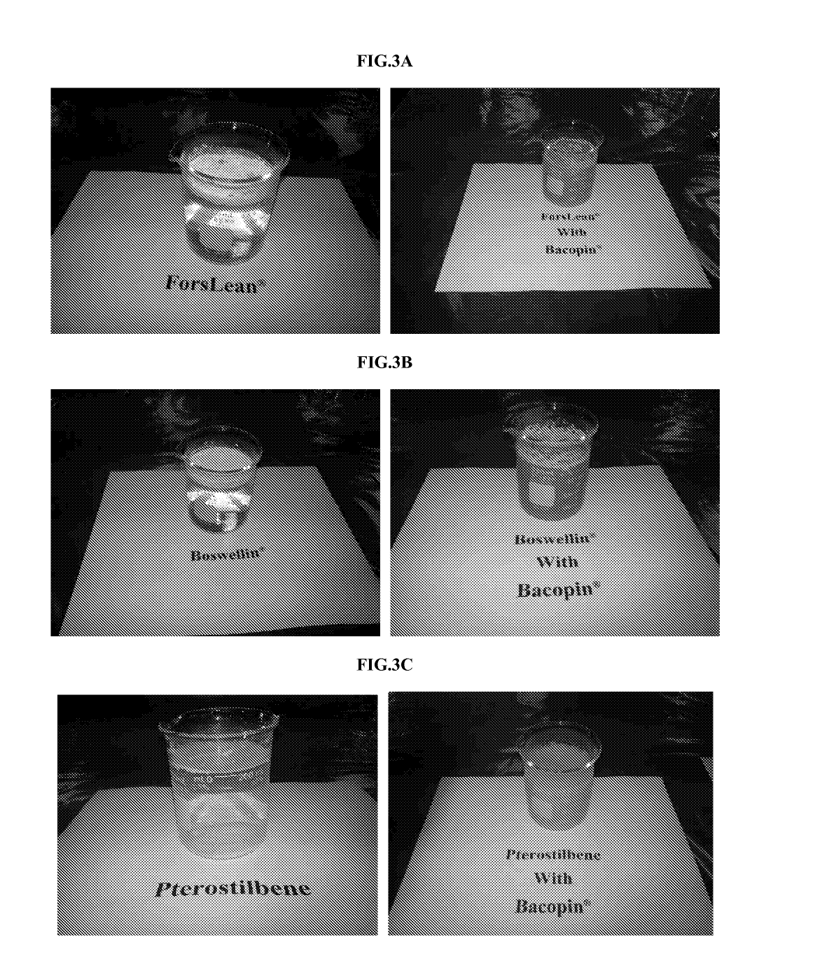

A. Five beakers each containing 200 ml distilled water at 37° C. were taken. To 3 beakers were added 2 gm of bioactive compounds forskolin (Forslean® 95%), boswellic acids (Boswellin® 70%) and pterostilbene 99% respectively. To the other 2 beakers were added 2 gm of Saw Palmetto extract 85% and Ginger Soft extract SCFE 35% respectively. The bioactive compounds/extracts either remained on the top, or precipitated to the bottom, or floated in the aqueous phase; but did not go into solution.
B. To each of the 5 beakers, Bacopa glycosides including 25% w/w to 75% w/w of total Bacosides (Bacopin®) was gradually added and mixed for 15 minutes until a homogenous emulsion was obtained. The Critical Micelle Concentration (CMC) for each system was determined (Table A; FIGS. 3A, 3B, 3C, 3D and 3E)

TABLE A

| | Bacopin 50%, | CMC % |
|---|---|---|
| Bioactive Compound (2 grams) | batch #H90481 | (wt/wt) |
| ForsLean 95%, lot no: J120096 | 0.4 gram | 20% |
| Boswellin 70%, lot no: H110465 | 0.88 gram | 44% |
| Pterostilbene 99%, lot no: C111167 | 0.29 gram | 14.50% |
| Saw Palmetto 85%, lot no: G100567 | 0.25 gram | 12.50% |
| Ginger Soft Extract-SCFE 35%, lot no: | 0.29 gram | 14.50% |
| SBX1248 | | |

### EXAMPLE 5

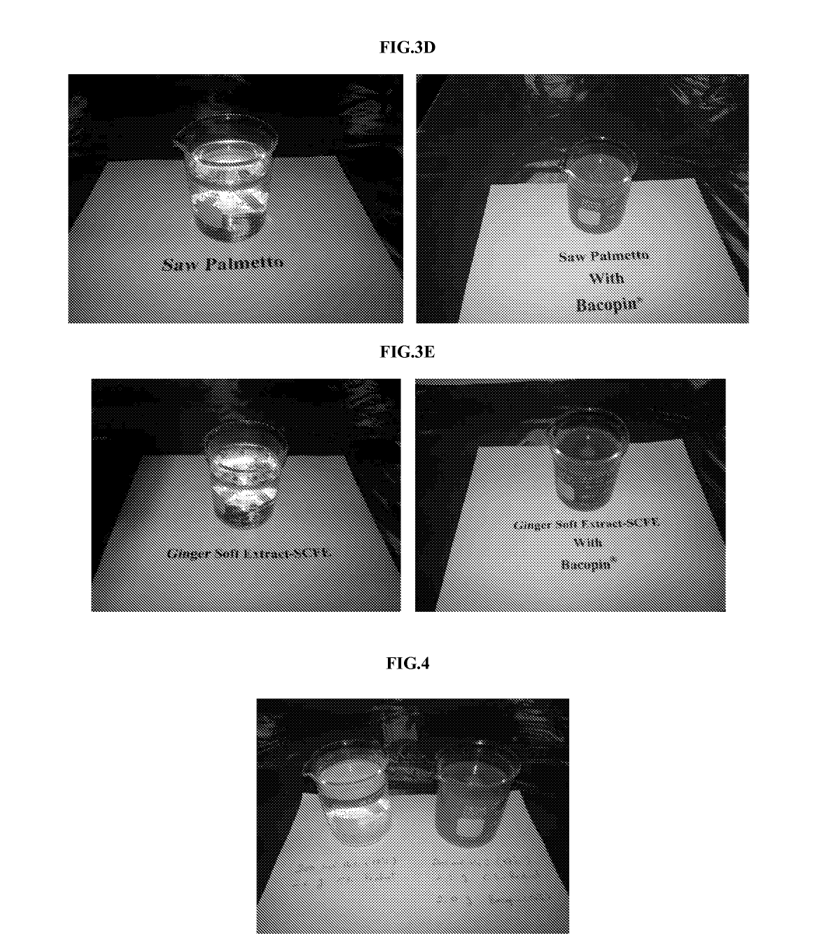

A. To a beaker containing 200 ml distilled water (37° C.) was added 2 grams of tetrahydrocurcuminoids (C3 Reduct®). The bioactive compound precipitated to the bottom of the beaker and did not go into solution.
B. Bacopa glycosides 25% w/w to 75% w/w of total Bacosides (Bacopin®) was gradually added up to 2 grams and mixed for 15 minutes. The majority (˜70%) of tetrahydrocurcuminoids was not observable at the bottom of the beaker anymore due to formation of emulsion (FIG. 4)

### EXAMPLE 6

As another exemplary embodiment, the present invention relates to a method of making 10%-50% w/w (HPLC) water soluble curcuminoids, said method comprising the steps of
A. Mixing 100 grams of triterpene glycosides with 5% 10% aqueous solution of PVP K-30 for 30 minutes under stirring at 60° C.;
B. Homogenously mixing the mixture of step A with 25% Curcuminoid mixture (Curcumin C3 Complex® from Sabinsa Corporation, NJ, USA), dissolved in ethanol under stirring at 70° C.;
C. Allowing the mixture of step B to settle overnight followed by filtering through silica based filter aids;
D. Stripping ethanol from the filtrate of step C under vacuum;
E. Adding 1% citric acid or ascorbic acid solution to the aqueous solution obtained in step D to adjust the pH range between 3-4;
F. Subjecting the aqueous solution of step E to spray drying/vacuum drying to obtain the final product. In one exemplary embodiment in which the triterpene glycosides used was Bacopa glycosides including 25% w/w to 75% w/w of total Bacosides (Bacopin®), the percentage composition ratio expressed as Curcuminoids: total bacosides: PVP K-30 was 10-15%:20%-30%:0%-1%.

Exemplary example of solubilization of insoluble bioactive compounds using triterpene glycosides in creating a galactogogue composition.

The composition may be formulated as follows.

| Chemical constituents | % composition |
|---|---|
| Shatavarin I-IV, quercetin and rutin- | 60-70 |
| Curcuminoids | |
| Lepidine and Leptidin-1 | 3-5 |
| Glycyrrhizinic | 3-5 |
| acid, Glabridin | |
| and Liquiritin | |
| L-Selenomethionine | 1-2 |
| Methyl | |
| selenocysteine | |
| and its | |
| dipeptides | |
| Excipients and Flavors | Quantum sufficient |

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method of solubilizing water-insoluble bioactive compounds and metabolites thereof using natural triterpene glycosides as solubilizing agents.

2. A method of solubilizing water-insoluble natural extracts using natural triterpene glycosides as solubilizing agents.

3. The method according to claims 1 and 2, wherein the triterpene glycosides are selected from the group comprising Shatavari glycosides obtained from Asparagus racemosus wherein Shatavari glycosides are standardized for not less than 4% w/w (HPLC) Shatavarin IV, Bacopa glycosides obtained from Bacopa monnieri wherein the Bacopa glycosides include 25% w/w to 75% w/w of total Bacosides and Centella glycosides from Centella Asiatica wherein the Centella glycosides include at least 8% w/w of total triterpenes.

4. A method of making 5%-50% w/w water soluble curcuminoids, said method comprising the steps of
A. Mixing 100 grams of triterpene glycosides with 5%-10% aqueous solution of PVP K-30 for 30 minutes under stirring at 60° C.;
B. Homogenously mixing the mixture of step A with 25% curcuminoid mixture, dissolved in ethanol under stirring at 70° C.;
C. Allowing the mixture of step B to settle overnight followed by filtering through Ceolite (Filteraid);
D. Stripping ethanol from the filtrate of step C under vacuum;
E. Adding 1% citric acid or ascorbic acid solution to the aqueous solution obtained in step D to adjust the pH range between 3-4;
F. Subjecting the aqueous solution of step E to spray drying/vacuum drying to obtain the final product.

5. A composition comprising water-insoluble bioactive compound and/or metabolites thereof solubilized by natural triterpene glycosides.

6. The composition according to claim 5 wherein water-insoluble bioactive compound and/or metabolites and triterpene glycosides occur in w/w ratios from 1:10 to 10:1.

7. A composition comprising water-insoluble natural extract solubilized using natural triterpene glycosides.

8. The composition according to claim 6 wherein the natural extract and triterpene glycosides are present in w/w ratios from 1:10 to 10:1.

9. The composition according to claims 5 and 7 wherein the natural triterpene glycosides are selected from the group comprising Shatavari glycosides obtained from Asparagus racemosus wherein Shatavari glycosides are standardized for not less than 4% w/w (HPLC) Shatavarin IV, Bacopa glycosides obtained from Bacopa monnieri wherein the Bacopa glycosides include 25% w/w to 75% w/w of total Bacosides and Centella glycosides from Centella Asiatica wherein the Centella glycosides include at least 8% w/w of total triterpenes.

10. The composition according to claims 5 and 7 wherein said composition further includes a bioavailability enhancing agent.

11. A galactogogue composition comprising curcuminoids solubilized using triterpene glycosides wherein said triterpene glycosides comprise Shatavari glycosides obtained from Asparagus racemosus standardized for not less than 4% w/w (HPLC) Shatavarin IV.
